(19) United States
(12) Patent Application Publication
(10) Pub. No.: US 2010/0323011 A1
(43) Pub. Date: Dec. 23, 2010

(54) PHARMACEUTICAL COMPOSITIONS OF A COMBINATION OF METFORMIN AND A DIPEPTIDYL PEPTIDASE-IV INHIBITOR

(76) Inventor: Nazaneen Pourkavoos, Ambler, PA (US)

Correspondence Address: MERCK, P O BOX 2000, RAHWAY, NJ, 07065-0907, US

(21) Appl. No.: 12/919,306

(22) PCT Filed: Feb. 23, 2009
(86) PCT No.: PCT/US09/34851

§ 371 Date: Aug. 25, 2010

Related U.S. Application Data
(60) Provisional application No. 61/068,016, filed on Mar. 4, 2008.

(51) Int. Cl. A61K 9/24; A61K 31/4985; A61P 3/10

(52) U.S. Cl. 424/465; 424/471; 514/249

(57) ABSTRACT

Disclosed are pharmaceutical compositions comprising fixed-dose combinations of an extended-release form of metformin, or a pharmaceutically acceptable salt thereof, coated with an immediate-release form of the DPP-4 inhibitor sitagliptin, or a pharmaceutically acceptable salt thereof.

### BACKGROUND OF THE INVENTION

Type 2 diabetes is a chronic and progressive disease arising from a complex pathophysiology involving the dual endocrine defects of insulin resistance and impaired insulin secretion. The treatment of Type 2 diabetes typically begins with diet and exercise, followed by oral antidiabetic monotherapy. For many patients, these regimens do not sufficiently control glycemia during long-term treatment, leading to a requirement for combination therapy within several years following diagnosis. However, co-prescription of two or more oral antidiabetic drugs may result in treatment regimens that are complex and difficult for many patients to follow. Combining two or more oral antidiabetic agents into a single tablet provides a potential means of delivering combination therapy without adding to the complexity of patients' daily regimens. Such formulations have been well accepted in other disease indications, such as hypertension (HYZAAR® which is a combination of losartan potassium and hydrochlorothiazide) and cholesterol lowering (VYTORIN® which is a combination of simvastatin and ezetimibe). The selection of effective and well-tolerated treatments is a key step in the design of a combination tablet. Moreover, it is essential that the components have complementary mechanisms of action and compatible pharmacokinetic profiles. Examples of marketed combination tablets containing two oral antidiabetic agents include Glucovance® (metformin and glyburide), Avandamet® (metformin and rosiglitazone), and Metaglip® (metformin and glipizide).

Metformin represents the only oral antidiabetic agent proven to reduce the total burden of microvascular and macrovascular diabetic complications and to prolong the lives of Type 2 diabetic patients. Furthermore, metformin treatment is often associated with reductions in body weight in overweight patients and with improvements in lipid profiles in dyslipidemic patients. Metformin hydrochloride is marketed in the U.S. and elsewhere as either immediate-release or extended-release formulations with tablet dosage strengths of 500, 750, 850, and 1000 milligrams. Extended-release formulations of metformin have advantages over immediate-release in terms of affording a more uniform maintenance of blood plasma active drug concentrations and providing better patient compliance by reducing the frequency of administration required.

Dipeptidyl peptidase-IV (DPP-4) inhibitors represent a new class of agents that are being developed for the treatment or improvement in glycemic control in patients with Type 2 diabetes. Specific DPP-4 inhibitors either already approved for marketing or under clinical development for the treatment of Type 2 diabetes include sitagliptin, vildagliptin, saxagliptin, melogliptin, P93/01 (Prosidion), alogliptin, denagliptin, Roche 0730699, TS021 (Taisho), and E3024 (Eisai). For example, oral administration of sitagliptin, vildagliptin, alogliptin, and saxagliptin to human Type 2 diabetics has been found to reduce fasting glucose and postprandial glucose excursion in association with significantly reduced HbA_{1c} levels. For reviews on the application of DPP-4 inhibitors for the treatment of Type 2 diabetes, reference is made to the following publications: (1) A. H. Stonehouse, et al., “Management of Type 2 diabetes: the role of incretin mimetics, Exp. Opin. Pharmacother., 7: 2095-2105 (2006); (2) B. D. Green, et al., “Inhibition of dipeptidyl peptidase-IV activity as a therapy of Type 2 diabetes,” Exp. Opin. Emerging Drugs, 11: 525-539 (2006); (3) M. M. J. Combettes, “GLP-1 and Type 2 diabetes: physiology and new clinical advances,” Curr. Opin. Pharmacol., 6: 598-605 (2006); and R. K. Campbell, “Rationale for Dipeptidyl Peptidase 4 Inhibitors: A New Class of Oral Agents for the Treatment of Type 2 Diabetes Mellitus,” Ann. Pharmacother., 41: 51-60 (2007).

Sitagliptin phosphate having structural formula I below is the dihydrogenphosphate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine.

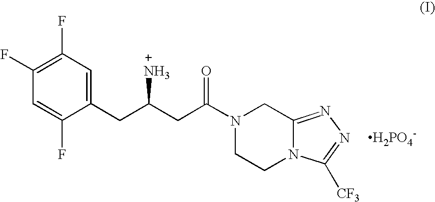

In one embodiment sitagliptin phosphate is in the form of a crystalline monohydrate. Sitagliptin free base and pharmaceutically acceptable salts thereof are disclosed in U.S. Pat. No. 6,699,871, the contents of which are hereby incorporated by reference in their entirety. Crystalline sitagliptin phosphate monohydrate is disclosed in U.S. Pat. No. 7,326,708, the contents of which are hereby incorporated by reference in their entirety. Sitagliptin phosphate has been approved for marketing in several countries, including the U.S., Europe, Canada, and Mexico, for the treatment of Type 2 diabetes and is branded as JANUVIA® in the U.S. and elsewhere. For reviews, see D. Drucker, et al., “Sitagliptin,” Nature Reviews Drug Discovery, 6: 109-110 (2007); C. F. Deacon, “Dipeptidyl peptidase 4 inhibition with sitagliptin: a new therapy for Type 2 diabetes,” Exp. Opin. Invest. Drugs, 16: 533-545 (2007); K. A. Lyseng-Williamson, “Sitagliptin,” Drugs, 67: 587-597 (2007); and B. Gallwitz, “Sitagliptin: Profile of a Novel DPP-4 Inhibitor for the Treatment of Type 2 Diabetes (Update),” Drugs of Today, 43: 801-814 (2007).

The combination of sitagliptin and metformin provides substantial and additive glycemic improvement in patients with Type 2 diabetes (B. J. Goldstein, et al., “Effect of Initial Combination Therapy with Sitagliptin, a DPP-4 Inhibitor, and Metformin on Glycemic Control in Patients with Type 2 Diabetes,” Diabetes Care, 30: 1979-1987 (2007) and B. Gallwitz, “Sitagliptin with Metformin: Profile of a combination for the treatment of Type 2 diabetes,” Drugs of Today, 43: 681-689 (2007). A fixed-dose combination of immediate-release of both metformin and sitagliptin has been approved for marketing in several countries, including U.S. and Mexico, for adult patients with Type 2 diabetes who are not adequately controlled on metformin or sitagliptin alone or in patients already being treated with the combination of sitagliptin and metformin. The combination is branded as JANUMET® in the U.S. JANUMET® tablets contain 50 mg sitagliptin and either 500, 850, or 1000 mg metformin. Pharmaceutical compositions comprising fixed-dose combinations of immediate-release sitagliptin and immediate-release metformin are disclosed in PCT international patent application WO 2007/078726 which published on Jul. 12, 2007.

Extended-release formulations of metformin are disclosed in U.S. Pat. No. 6,340,475; U.S. Pat. No. 6,635,280; U.S. Pat. No. 6,866,866; U.S. Pat. No. 6,475,521; and U.S. Pat. No. 6,660,300. Pharmaceutical formulations containing extended-release metformin and a thiazolidinedione antihyperglycemic agent are described in WO 2004/026241 (1 Apr. 2004) and WO 2006/107528 (12 Oct. 2006). Pharmaceutical compositions comprising a DPP-4 inhibitor and a slow-release form of metformin are disclosed in US 2007/0172525 (26 Jul. 2007). Stable pharmaceutical compositions of an immediate-release form of the antihyperglycemic sulfonylurea glimepiride and extended-release metformin are disclosed in US 2007/0264331 (15 Nov. 2007).

The present invention provides for pharmaceutical compositions comprising a core tablet formulation of a fixed-amount of metformin that is coated with a sustained-release (SR) polymer film which is further coated with an immediate release form of a fixed amount of sitagliptin. The metformin core tablet is prepared by wet or dry processing methods prior to coating with the SR polymer composition.

The present invention also provides processes to prepare pharmaceutical compositions of a fixed-dose combination of immediate-release sitagliptin and extended-release metformin by wet or dry processing methods. The wet processing methods include wet granulation.

Another aspect of the present invention provides methods for the treatment of Type 2 diabetes by administering to a host in need of such treatment a therapeutically effective amount of a pharmaceutical composition of the present invention.

These and other aspects of the invention will become readily apparent from the detailed description which follows.

### SUMMARY OF THE INVENTION

The present invention is directed to novel pharmaceutical compositions comprising a core tablet formulation of metformin, or a pharmaceutically acceptable salt thereof, coated with a sustained-release polymer film which is further coated with an immediate-release form of the DPP-4 inhibitor sitagliptin, or a pharmaceutically acceptable salt thereof, processes for preparing such compositions, and methods of treating Type 2 diabetes with such compositions. In particular, the invention is directed to pharmaceutical compositions comprising a core tablet formulation of metformin hydrochloride coated with a sustained-release polymer film which is further coated with an immediate-release form of sitagliptin phosphate.

### BRIEF DESCRIPTION OF THE FIGURES

FIG. 1 is a graph showing in vitro metformin dissolution profiles of an immediate-release (IR) 1000-mg metformin hydrochloride core tablet coated with cellulose acetate sustained-release polymer film compositions of varying porosity with 3, 5, or 7 weight percent gain relative to the core tablet weight.

FIG. 2 is a graph comparing in vitro metformin dissolution profiles of an immediate-release (IR) 500-mg metformin hydrochloride tablet with metformin dissolution profiles of an immediate-release (IR) 1000-mg metformin hydrochloride core tablet coated with a high porosity cellulose acetate sustained-release polymer film composition with 3, 5, or 7 weight percent gain relative to the core tablet weight.

FIG. 3 is a graph comparing in vitro metformin dissolution profiles of an immediate-release (IR) 500-mg metformin hydrochloride tablet with metformin dissolution profiles of a 1000-mg immediate-release (IR) metformin hydrochloride core tablet coated with a “modified high porosity” cellulose acetate sustained-release polymer film composition with 3, 5, or 7 weight percent gain relative to the core tablet weight.

FIG. 4 is a graph showing in vitro dissolution profiles for sitagliptin phosphate from the drug film layer in a pharmaceutical composition of the present invention compared to sitagliptin phosphate in JANUMET™ which is a marketed fixed-dose combination of immediate-release metformin hydrochloride and immediate-release sitagliptin phosphate.

### DETAILED DESCRIPTION OF THE INVENTION

One aspect of the present invention is directed to pharmaceutical compositions comprising a core tablet formulation of a fixed-amount of metformin, or a pharmaceutically acceptable salt thereof, which core tablet is coated with a sustained-release polymer film which is further coated with an immediate release form of a fixed amount of the DPP-4 inhibitor sitagliptin, or a pharmaceutically acceptable salt thereof.

A preferred pharmaceutically acceptable salt of sitagliptin is the dihydrogenphosphate salt of structural formula I above (sitagliptin phosphate). A preferred form of the dihydrogenphosphate salt is the crystalline monohydrate disclosed in U.S. Pat. No. 7,326,708, the contents of which are hereby incorporated by reference in their entirety.

The preparation of sitagliptin, and pharmaceutically acceptable salts thereof, is disclosed in U.S. Pat. No. 6,699,871, the contents of which are herein incorporated by reference in their entirety. The preparation of sitagliptin. phosphate monohydrate is disclosed in U.S. Pat. No. 7,326,708, the contents of which are hereby incorporated by reference in their entirety.

The unit dosage strength of sitagliptin free base anhydrate (active moiety) for inclusion into the fixed-dose combination pharmaceutical compositions of the present invention is 25, 50, and 100 milligrams. An equivalent amount of sitagliptin phosphate monohydrate to the sitagliptin free base anhydrate is used in the pharmaceutical compositions, namely, 32.125, 64.25 and 128.5 milligrams, respectively.

The unit dosage strength of the metformin hydrochloride for incorporation into the fixed-dose combination of the present invention is 250, 500, 750, 850, and 1000 milligrams. These unit dosage strengths of metformin hydrochloride represent the dosage strengths approved in the U.S. for marketing to treat Type 2 diabetes.

Specific embodiments of dosage strengths for sitagliptin and metformin hydrochloride in the fixed-dose combinations of the present invention are the following:
(1) 25 milligrams of sitagliptin (equivalent to 32.125 milligrams of sitagliptin phosphate monohydrate) and 250 milligrams metformin hydrochloride;
(2) 25 milligrams of sitagliptin (equivalent to 32.125 milligrams of sitagliptin phosphate monohydrate) and 500 milligrams metformin hydrochloride;
(3) 25 milligrams of sitagliptin (equivalent to 32.125 milligrams of sitagliptin phosphate monohydrate) and 750 milligrams metformin hydrochloride;
(4) 25 milligrams of sitagliptin (equivalent to 32.125 milligrams of sitagliptin phosphate monohydrate) and 850 milligrams metformin hydrochloride;
(5) 25 milligrams of sitagliptin (equivalent to 32.125 milligrams of sitagliptin phosphate monohydrate) and 1000 milligrams metformin hydrochloride;
(6) 50 milligrams of sitagliptin (equivalent to 64.25 milligrams of sitagliptin phosphate monohydrate) and 500 milligrams metformin hydrochloride;
(7) 50 milligrams of sitagliptin (equivalent to 64.25 milligrams of sitagliptin phosphate monohydrate) and 750 milligrams metformin hydrochloride;
(8) 50 milligrams of sitagliptin (equivalent to 64.25 milligrams of sitagliptin phosphate monohydrate) and 850 milligrams metformin hydrochloride;
(9) 50 milligrams of sitagliptin (equivalent to 64.25 milligrams of sitagliptin phosphate monohydrate) and 1000 milligrams metformin hydrochloride;
(10) 100 milligrams of sitagliptin (equivalent to 128.5 milligrams of sitagliptin phosphate monohydrate) and 500 milligrams metformin hydrochloride;
(11) 100 milligrams of sitagliptin (equivalent to 128.5 milligrams of sitagliptin phosphate monohydrate) and 750 milligrams metformin hydrochloride;
(12) 100 milligrams of sitagliptin (equivalent to 128.5 milligrams of sitagliptin phosphate monohydrate) and 850 milligrams metformin hydrochloride; and
(13) 100 milligrams of sitagliptin (equivalent to 128.5 milligrams of sitagliptin phosphate monohydrate) and 1000 milligrams metformin hydrochloride.

In a particular aspect of the present invention, the pharmaceutical compositions of the present invention comprise an inner core formulation of metformin hydrochloride. The formulation is compressed into a tablet form.

The metformin core tablets are prepared by wet or dry processing methods. In one embodiment the metformin core tablets are prepared by wet processing methods. In a class of this embodiment the metformin core tablets are prepared by wet granulation methods. With wet granulation either high-shear granulation or fluid-bed granulation is preferred, but other wet granulation methods may also be used.

In the high-shear wet granulation process, metformin hydrochloride is first blended with a suitable binding agent using water or an aqueous alcohol mixture, such as aqueous ethanol, as the granulating solvent. In one embodiment the high-shear granulation process uses a tip speed of 3.58 msec with a granulation fluid level of between 3 and 10%. The resulting granules are next dried and sized to produce a mean particle size range of about 500 to about 800 microns and have a tensile strength of about 2 to about 3 megapascals [MPa] over a compaction pressure range of about 200 to 400 MPa. Embodiments of suitable binding agents include hydroxypropylcellulose (HPC), hydroxypropylmethyl cellulose (HMPC), hydroxyethyl-cellulose, starch 1500, polyvinylpyrrolidone (povidone), and co-povidone. A preferred binding agent is polyvinylpyrrolidone.

The sized metformin granulation is subsequently blended with an extragranular composition which consists of one or more diluents and optionally a suitable glidant and/or a suitable lubricant to afford a final metformin drug loading of about 50 to about 80 weight percent. The tensile strength of the final blend formulation is about 2.0 MPa to about 2.5 MPa over a range of about 200 MPa to about 400 MPa compaction pressure. The final blend is compressed on a rotary press at a compression force of about 30 kiloNewtons (kN) using modified capsule-shaped tooling resulting in a tablet hardness (breaking force) of about 30-35 kiloponds (kp).

Embodiments of diluents include, but are not limited to, mannitol, sorbitol, dibasic calcium phosphate dihydrate, microcrystalline cellulose, and powdered cellulose. A preferred diluent is microcrystalline cellulose. Microcrystalline cellulose is available from several suppliers and includes Avicel PH 101™, Avicel PH 102™, Avicel PH 103™, Avicel PH 105™, and Avicel PH 200™, manufactured by the FMC Corporation.

Examples of lubricants include magnesium stearate, calcium stearate, stearic acid, sodium stearyl fumarate, hydrogenated castor oil, and mixtures thereof. A preferred lubricant is magnesium stearate or sodium stearyl fumarate or a mixture thereof. Examples of glidants include colloidal silicon dioxide, calcium phosphate tribasic, magnesium silicate, and talc. In one embodiment the glidant is colloidal silicon dioxide and the lubricant is sodium stearyl fumarate.

The composition of a representative metformin core tablet of the present invention is provided in Table 1.

TABLE 1
Metformin Core Tablet Composition

| | | Final drug loading |
|---|---|---|
| Component | Granulation | (% w/w) |
| Metformin HCl | 93.0% | 76.725 |
| PVP K 29/32 | 7.0% | 5.775 |
| Intragranular | 100.0% | |
| Weight | | |
| Avicel PH 102 ™ | | 15.0 |
| Colloidal silicon | | 0.50 |
| dioxide | | |
| Sodium stearyl | | 2.0 |
| fumarate | | |
| Total | | 100 |

In a second aspect of the present invention, the metformin core tablet is coated with a functional sustained-release (SR) polymer film that is designed to control the release of metformin from the soluble core tablet leaving a largely intact ghost polymer shell. The polymer film is designed as a porous membrane. The sustained-release polymer film consists of an aqueous organic solution of a sustained-release (SR) polymer, one or more plasticizers, and a pore-forming agent. In one embodiment, the aqueous organic solvent is aqueous acetone.

Embodiments of sustained-release polymers are cellulose esters, cellulose diesters, cellulose triesters, cellulose ethers, mixed cellulose esters/ethers, ethylcellulose having viscosity grades from 10 to 50 cP, ethylcellulose aqueous dispersion, polyvinyl acetate, and methacrylic acid copolymers. In one embodiment, the sustained-release polymer is a cellulose ester selected from the group consisting of cellulose acetate, cellulose diacetate, cellulose triacetate, cellulose acetate propionate, and cellulose acetate butyrate. In a subclass of this class the sustained-release polymer is cellulose acetate. In a subclass of this subclass the cellulose acetate is cellulose acetate (CA) having an acetyl content of about 39.8 weight percent as in the CA-398-10 which is commercially available from Eastman Fine Chemicals.

Embodiments of plasticizers include, but are not limited to, dibutyl sebacate, diethyl phthalate, triethyl citrate, tri-n-butyl citrate, acetyl tri-n-butyl citrate, acetylated monoglycerides, castor oil, olive oil, sesame oil, oleic acid, and triacetin (glyceryl triacetate). In a particular class the plasticizer is triacetin.

Embodiments of pore-forming agents include, but are not limited to, sodium chloride, potassium chloride, sucrose, sorbitol, mannitol, polyethylene glycols (PEG), propylene glycol, polyvinyl alcohols, and methacrylic acid copolymers. In one embodiment the polyethyleneglycol is PEG 3350. In a particular class the SR polymer is cellulose acetate and the plasticizer is triacetin.

The amount of sustained-release polymer coated over the metformin core tablet is based on the percent weight gain and ranges from about 1 to about 10 weight percent. The total concentration of solids (SR polymer+plasticizer+pore-forming agent) in the aqueous organic solution is preferably kept at about 10 weight percent. The ratio of the organic solvent to water is about 3:1 (w/w). The percent level of plasticizers to cellulose acetate ranges from about 25 to about 150 weight percent resulting in low to high porosity membrane coatings to modulate the rate of metformin drug release. In one embodiment the amount of sustained-release polymer coated over the metformin core tablet is based on the percent weight gain and ranges from about 3 to about 9 weight percent. In a class of this embodiment the amount of sustained-release polymer coated over the metformin core tablet ranges from about 3 to about 7 weight percent.

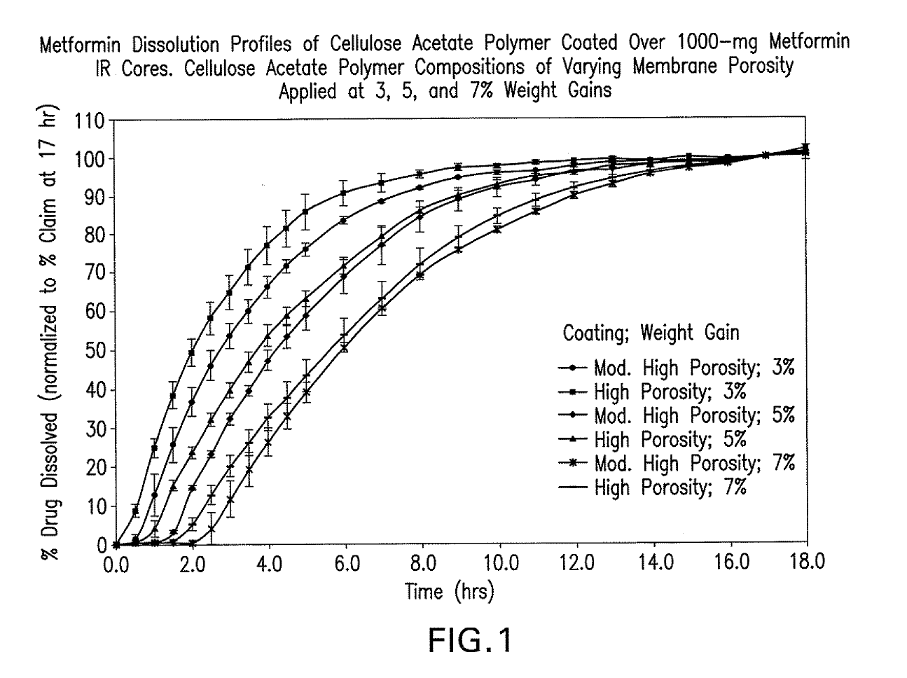

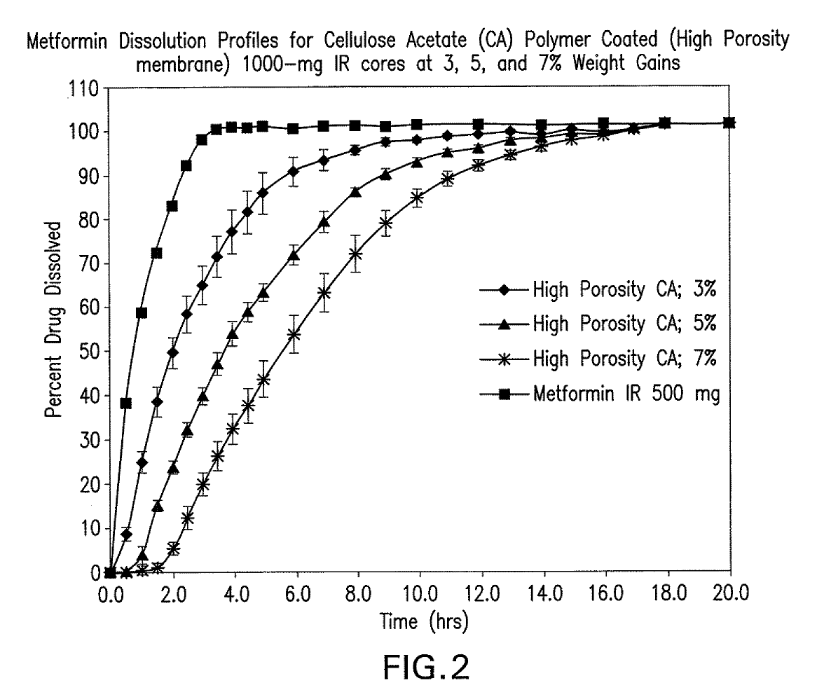

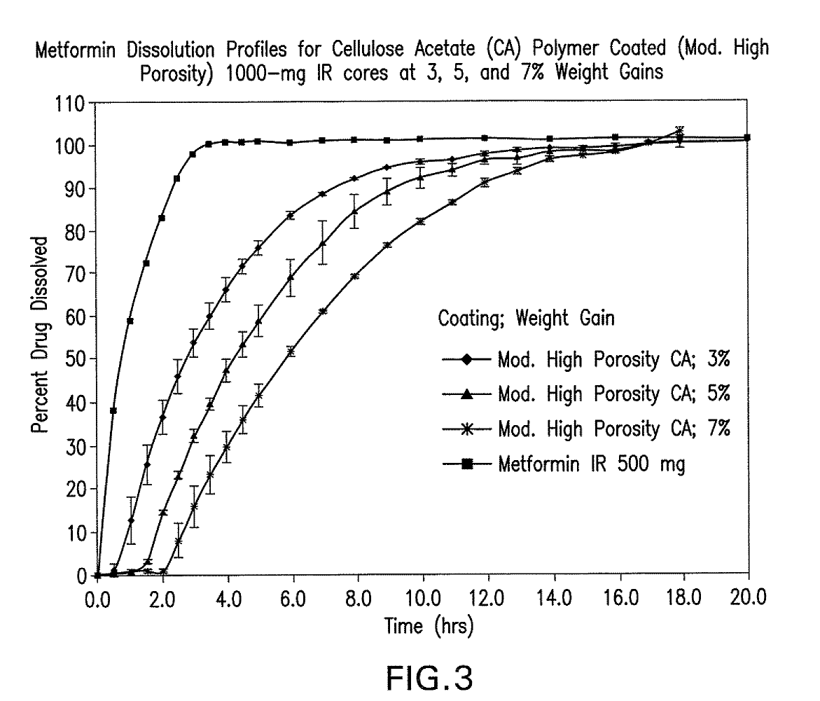

The composition of representative sustained-release (SR) cellulose acetate polymer films of different porosities from low to high is provided in Table 2. The SR polymer coating solution is prepared with differing levels of cellulose acetate (4-8 weight percent of CA) and a 1:1 w/w ratio of triacetin and PEG 3350. The total solid concentration is kept the same as well as the ratio of acetone to water. The modified high porosity composition (5 weight percent of CA) generally affords a more robust film in terms of processability and integrity of polymer. The cellulose acetate polymer solution is applied at various levels of weight gain ranging from about 3 to about 9 weight percent based on core tablet weight and results in different rates of metformin drug release as shown in the metformin in vitro dissolution profiles of FIGS. 1-3.

TABLE 2
Sustained-Release Polymer Film Composition*

| | | Modified | | |
|---|---|---|---|---|
| | High | High | Medium | Low |
| | Porosity | Porosity | Porosity | Porosity |
| Component | % w/w | % w/w | % w/w | % w/w |
| CA-398-10** | 4 | 5 | 6 | 8 |
| PEG 3350 | 3.0 | 2.5 | 2 | 1 |
| Triacetin | 3.0 | 2.5 | 2 | 1 |
| Acetone | 68 | 68 | 68 | 68 |
| Water | 22 | 22 | 22 | 22 |
| Total | 100 | 100 | 100 | 100 |

*10% solid concentration (CA + PEG 3350 + triacetin).
**Grade of commercial cellulose acetate having an acetyl content of about 39.8 weight percent.

In one embodiment the cellulose acetate aqueous organic coating solution is applied over the metformin core tablet to achieve weight gain of about 3 to about 9 percent resulting in variable metformin release profiles using the high to modified high porosity compositions shown in Table 2. The film coating of cellulose acetate polymer is carried out in a conventional perforated vented pan with baffles and is conducted at a controlled exhaust temperature range of about 25 to 35° C.

In a third aspect of the present invention, the SR coated metformin core tablet is further coated with an aqueous solution or suspension of a sitagliptin salt until the desired solid weight gain, typically corresponding to either 50 mg or 100 mg of sitagliptin, is obtained.

The sitagliptin coating solution or suspension is designed to produce a stable solution in an immediate-release polymer film so that the drug is substantially present as an amorphous form to allow rapid dissolution and absorption of sitagliptin to take place following ingestion of the dosage form. Embodiments of the film-forming polymer are hydroxypropylmethylcellulose (HPMC), hydroxypropylcellulose (HPC), sodium carboxymethylcellulose, polyvinylpyrrolidone (PVP), and polyvinylalcohol/PEG 3350. A particular form of HPMC for use as a film-forming polymer is HPMC 2910. The coating solution also optionally contains one or more excipients selected from the group consisting of a plasticizer, such as polyethylene glycol grades 400 to 3350 and triethyl citrate; a dispersing agent, such as hydrated aluminum silicate (Kaolin); a colorant; and an antioxidant to prevent oxidative degradation. The antioxidant is selected from the group consisting of α-tocopherol, γ-tocopherol, δ-tocopherol, extracts of natural origin rich in tocopherol, L-ascorbic acid and its sodium or calcium salts, ascorbyl palmitate, propyl gallate, octyl gallate, dodecyl gallate, butylated hydroxytoluene (BHT), and butylated hydroxyanisole (BHA). In one embodiment, the antioxidant is propyl gallate.

The sitagliptin coating solution or suspension is prepared in total concentration of about 12 to about 17 weight percent. The sitagliptin coating solution or suspension is applied to the metformin core tablet and the amount of sitagliptin phosphate deposited in the active pharmaceutical ingredient (“API”) film layer is controlled by tablet weight gain or amount of coating suspension sprayed. The 50 mg sitagliptin phosphate film potency represents one-half the weight gain of the 100 mg potencies.

The composition of a representative sitagliptin film coating solution or suspension is provided in Table 3.

TABLE 3
Sitagliptin Aqueous Film Coating Solution Compositions

| | Solid Concentration | Solid Concentration |
|---|---|---|
| Ingredient | at about 12% (w/w) | at about 17% (w/w) |
| Sitagliptin phosphate | 6.0 | 12.0 |
| monohydrate | | |
| Opadry I Clear | | 5.0 |
| HPMC 2910 (6 cP) | 3.75 | |
| PEG 3350 NF | 0.75 | |
| Kaolin (Compendial) | 1.5 | |
| Propyl gallate | 0.0637 | 0.0637 |
| FD& C blue lake dye | 0.10 | |
| Water | 87.84 | 82.936 |
| To Make | 100 | 100 |

The film-coating operation is carried out in a conventional perforated vented pan with baffles and is conducted at a controlled exhaust temperature range of about 40° C. to about 44° C. The spray rate and air flow through the coating pan is adjusted to produce a uniform coating and coverage of the entire width of the tablet bed. The amount of the coating solution or suspension applied is controlled by percent weight gain of tablet cores and typically ranges from about 19 to about 22 weight percent. This range results in sitagliptin drug assay close to the desired 50 mg or 100 mg with a standard deviation of about 24% for content uniformity assay of sitagliptin. The duration of the coating step is about 4-7 hours but may vary depending on the type of equipment used.

The final pharmaceutical compositions of the present invention are tablets. The tablets may be further film-coated such as with a mixture of hydroxypropylcellulose and hydroxypropylmethylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; or any other suitable immediate-release film-coating agent(s). A commercial film-coat is Opadry® which is a formulated powder blend provided by Colorcon.

The pharmaceutical tablet compositions of the present invention may also contain one or more additional formulation ingredients selected from a wide variety of excipients known in the pharmaceutical formulation art. According to the desired properties of the pharmaceutical composition, any number of ingredients may be selected, alone or in combination, based upon their known uses in preparing tablet compositions. Such ingredients include, but are not limited to, diluents, compression aids, glidants, disintegrants, lubricants, flavors, flavor enhancers, sweeteners, and preservatives.

The term “tablet” as used herein is intended to encompass compressed pharmaceutical dosage formulations of all shapes and sizes, whether coated or uncoated.

In one embodiment the metformin core tablets are prepared by wet granulation (preferably high shear and/or fluid bed). The steps involved in the wet granulation method comprise the following:
(1) the active pharmaceutical ingredient metformin hydrochloride is added to the granulator bowl;
(2) optional disintegrants are added to step 1;
(3) for high-shear granulation, the binding agent (such as polyvinylpyrrolidone or hydroxypropylcellulose) is added dry to the granulator bowl and dry mixed for a short period followed by the addition of water with or without a surfactant (such as sodium lauryl sulfate); for fluid bed granulation, the metformin hydrochloride is added to the granulator bowl, the powder is fluidized, and the granulating solution comprised of binding agent with or without surfactant in water is sprayed into the fluidized powder;
(4) granules prepared by high-shear granulation are tray-dried in an oven or dried in a fluid bed dryer. For granules prepared by fluid-bed granulation, granules are dried in a fluid bed dryer;
(5) dried granules are resized using a suitable mill;
(6) optional diluents (such as microcrystalline cellulose and dibasic calcium phosphate dihydrate) are blended with dried and sized granules in a suitable blender;
(7) lubricants or glidants (such as magnesium stearate and sodium stearyl fumarate) are added to the blend from step 7 in a suitable blender; and
(8) the lubricated granule mixture from step 8 is compressed into the desired tablet image.

The present invention also provides methods for treating Type 2 diabetes by orally administering to a host in need of such treatment a therapeutically effective amount of one of the fixed-dose combination pharmaceutical compositions of the present invention. In one embodiment the host in need of such treatment is a human. In another embodiment the pharmaceutical composition is in the dosage form of a tablet. The pharmaceutical compositions comprising the fixed-dose combination may be administered once-daily (QD) or twice-daily (BID).

The following examples further describe and demonstrate embodiments within the scope of the present invention. The examples are given solely for the purpose of illustration and are not intended to be construed as limitations of the present invention as many variations thereof are possible without departing from the spirit and scope of the invention.

### Example 1

### Fixed-Dose Combination of 50 or 100 Milligrams of Sitagliptin and 1000 Milligrams of Metformin Hydrochloride Coated with Sustained-Release Polymer (3% w/w Level)

| | 100/1000 mg/ | | 50/1000 mg/ | |
|---|---|---|---|---|
| Ingredient | tablet | 100/1000% w/w | tablet | 50/1000% w/w |
| 1. Tablet Core | | | | |
| Metformin HCl | 1000 | 76.725 | 1000 | 76.725 |
| PVP K29/32 | 75.27 | 5.775 | 75.27 | 5.775 |
| Avicel PH 102 ™ | 195.50 | 15 | 195.50 | 15 |
| Silicon Dioxide | 6.517 | 0.5 | 6.517 | 0.5 |
| Sodium stearyl fumarate | 26.067 | 2.0 | 26.067 | 2.0 |
| Total Tablet cores | 1303.36 | 100 | 1303.36 | 100 |
| 2. Cellulose Acetate (CA) | | | | |
| Polymer Coating | | | | |
| CA-398-10 | 19.55 | 1.5 | 19.55 | 1.5 |
| PEG 3350 | 9.775 | 0.75 | 9.775 | 0.75 |
| Triacetin | 9.775 | 0.75 | 9.775 | 0.75 |
| Total CA SR coat | 39.1 | 3 | 39.1 | 3 |
| SR Coated Tablets | 1342.46 | 103 | 1342.46 | 103 |
| 3. Sitagliptin Coating | | | | |
| Sitagliptin phosphate | 128.52* | 9.57 | 64.26** | 4.79 |
| monohydrate | | | | |
| Propyl gallate | 1.36 | 0.101 | 0.68 | 0.05 |
| HPMC/PEG/Kaolin/dye | 130.66 | 9.73 | 65.33 | 4.87 |
| Total Sitagliptin Coat | 260.55 | 19.41 | 130.27 | 9.70 |
| Total Coated Tablet | 1603.01 | 122.41 | 1472.74 | 112.7 |

*Equivalent to 100 mg of sitagliptin free base anhydrate.
**Equivalent to 50 mg of sitagliptin free base anhydrate.

### Steps in the Preparation of Example 1:

(1) metformin hydrochloride was delumped by passing it through a suitable mill;
(2) the delumped metformin and PVP dry binder powder were transferred into a granulator bowl of a high-shear granulator and granulated with water at a level of 3 to 10% of total dry powder batch size until granules were formed;
(3) the granules were dried in an oven at 50° C. to a moisture content of less than 2%;
(4) the dried granules were sized in a suitable mill to obtain a mean granule particle size of about 500-800 microns;
(5) the dried and sized granules were blended with microcrystalline cellulose (Avicel PH 102) and pre-screened (mesh #20) silicon dioxide;
(6) the pre-screened (mesh #60) sodium stearyl fumarate and blend from step 5 were blended in a suitable blender to produce the final blend;
(7) the final blend from step 6 was compressed in a rotary tablet press at a main compression force of about 30 kN to produce tablets at the target weight range and hardness;
(8) the sustained-release polymer coating solution was prepared by first dissolving the cellulose acetate polymer in the acetone water mixture, and then adding the PEG 3350 and triacetin to the solution while mixing until all solids were dissolved;
(9) the compressed tablet cores from step 7 were loaded into a suitable perforated side-vented coating pan with baffles fitted with single or multiple spray guns to produce a spray fan to uniformly cover the tablet bed;
(10) the tablet bed was warmed in the rotating coating pan until an exhaust temperature of 25-35° C. was reached at an inlet air flow of about 28-42 cubic feet/min (CFM);
(11) the average weight of warmed uncoated tablet was determined as the initial starting weight;
(12) the cellulose acetate coating solution was sprayed onto the tablet bed at a suitable spray rate and atomization pressure;
(13) spraying with the cellulose acetate polymer coating solution was continued while monitoring the tablet weight until the required weight gain was obtained; an approximate dried polymer coat weight of 39 mg was deposited over the tablet cores;
(14) spraying was stopped, and the tablets were dried and discharged from the coating pan;
(15) the sitagliptin phosphate coating solution was prepared by mixing all the excipients (except Kaolin) and sitagliptin phosphate in the required amount of purified water using a suitable homogenizer until the solids were dissolved;
(16) the pre-screened (mesh #60) Kaolin powder was added to the sitagliptin phosphate coating solution and mixed with a suitable mixer and blade until the powder was uniformly dispersed in the coating solution;
(17) the compressed tablet cores from step 7 were loaded into a suitable perforated side-vented coating pan with baffles fitted with single or multiple spray guns to produce a spray fan to cover the entire width of the tablet bed;
(18) the tablet bed was warmed in the rotating coating pan until an exhaust temperature of 40-44° C. was reached at an inlet air flow of about 270-350 cubic feet/min (CFM);
(19) the average weight of warmed uncoated tablet was determined as the initial starting weight;
(20) the sitagliptin phosphate coating dispersion was sprayed onto the tablet bed at a suitable spray rate and atomization pressure;
(21) spraying of the sitagliptin phosphate coating dispersion was continued while monitoring the tablet weight until the required weight gain was obtained;
(22) an approximate dried coat weight of 130 mg equivalent to 50 mg sitagliptin (as free base) or 260 mg equivalent to 100 mg of sitagliptin (as free base) was deposited over the tablet cores; and
(23) spraying was stopped, and the tablets were dried and discharged from the coating pan.

### Example 2

### Fixed-Dose Combination of 50 or 100 Milligrams of Sitagliptin and 1000 Milligrams of Metformin Hydrochloride Coated with Sustained-Release Polymer (5% w/w)

| | 100/1000 mg/ | | 50/1000 mg/ | |
|---|---|---|---|---|
| Ingredient | tablet | 100/1000% w/w | tablet | 50/1000% w/w |
| 1. Tablet Core | | | | |
| Metformin HCl | 1000 | 76.725 | 1000 | 76.725 |
| PVP K29/32 | 75.27 | 5.775 | 75.27 | 5.775 |
| Avicel PH 102 ™ | 195.50 | 15 | 195.50 | 15 |
| Silicon Dioxide | 6.517 | 0.5 | 6.517 | 0.5 |
| Sodium stearyl fumarate | 26.067 | 2.0 | 26.067 | 2.0 |
| Total Tablet cores | 1303.36 | 100 | 1303.36 | 100 |
| 2. Cellulose Acetate (CA) | | | | |
| Polymer Coating | | | | |
| CA-398-10 | 32.58 | 2.5 | 32.58 | 2.5 |
| PEG 3350 | 16.29 | 1.25 | 16.29 | 1.25 |
| Triacetin | 16.29 | 1.25 | 16.29 | 1.25 |
| Total CA SR coat | 65.16 | 5 | 65.16 | 5 |
| SR Coated Tablets | 1368.42 | 105 | 1368.42 | 105 |
| 3. Sitagliptin Coating | | | | |
| Sitagliptin phosphate | 128.52* | 9.39 | 64.26** | 4.70 |
| monohydrate | | | | |
| Propyl gallate | 1.36 | 0.10 | 0.68 | 0.05 |
| HPMC/PEG/Kaolin/dye | 130.66 | 9.55 | 65.33 | 4.77 |
| Total Sitagliptin Coat | 260.55 | 19.04 | 130.27 | 9.52 |
| Total Coated Tablet | 1628.97 | 124.04 | 1498.69 | 114.52 |

*Equivalent to 100 mg of sitagliptin free base anhydrate.
**Equivalent to 50 mg of sitagliptin free base anhydrate.

### Steps in Preparation of Example 2:

(1) metformin hydrochloride was delumped by passing it through a suitable mill;
(2) the delumped metformin and PVP dry binder powder were transferred into a granulator bowl of a high-shear granulator and granulated with water at a level of 3 to 10% of total dry powder batch size until granules were formed;
(3) the granules were dried in an oven at 50° C. to a moisture content of less than 2%;
(4) the dried granules were sized in a suitable mill to obtain a mean granule particle size of about 500-800 microns;
(5) the dried and sized granules were blended with microcrystalline cellulose (Avicel PH 102) and pre-screened (mesh #20) silicon dioxide;
(6) the pre-screened (mesh #60) sodium stearyl fumarate and blend from step 5 were blended in a suitable blender to produce the final blend;
(7) the final blend from step 6 was compressed in a rotary tablet press to produce tablets at the target weight range and hardness;
(8) the organic polymer solution was prepared by first dissolving the cellulose acetate polymer in the acetone water mixture, and then adding the PEG 3350 and triacetin to the solution while mixing until all solids were dissolved;
(9) the compressed tablet cores from step 7 were loaded into a suitable perforated side-vented coating pan with baffles fitted with single or multiple spray guns to produce a spray fan to uniformly cover the tablet bed;
(10) the tablet bed was warmed in the rotating coating pan until an exhaust temperature of 25-35° C. was reached;
(11) the average weight of warmed uncoated tablet was determined as the initial starting weight;
(12) the cellulose acetate coating solution was sprayed onto the tablet bed at a suitable spray rate and atomization pressure;
(13) spraying with the cellulose acetate polymer coating solution was continued while monitoring the tablet weight until the required weight gain was obtained; an approximate dried polymer coat weight of 65 mg was deposited over the tablet cores;
(14) spraying was stopped, and the tablets were dried and discharged from the coating pan;
(15) the sitagliptin phosphate coating solution was prepared by mixing all the excipients (except Kaolin) and sitagliptin phosphate in the required amount of purified water using a suitable homogenizer until the solids were dissolved;
(16) the pre-screened (mesh 460) Kaolin powder was added to the sitagliptin phosphate coating solution and mixed with a suitable mixer and blade until the powder was uniformly dispersed in the coating solution;
(17) the compressed tablet cores from step 7 were loaded into a suitable perforated side-vented coating pan with baffles fitted with single or multiple spray guns to produce a spray fan to cover the entire width of the tablet bed;
(18) the tablet bed was warmed in the rotating coating pan until an exhaust temperature of 40-44° C. was reached;
(19) the average weight of warmed uncoated tablet was determined as the initial starting weight;
(20) the sitagliptin phosphate coating dispersion was sprayed onto the tablet bed at a suitable spray rate and atomization pressure;
(21) spraying of the sitagliptin phosphate coating dispersion was continued while monitoring the tablet weight until the required weight gain was obtained;
(22) an approximate dried coat weight of 130 mg equivalent to 50 mg sitagliptin (as free base) or 260 mg equivalent to 100 mg of sitagliptin (as free base) was deposited over the tablet cores; and
(23) spraying was stopped, and the tablets were dried and discharged from the coating pan.

### Example 3

### Fixed-Dose Combination of 50 or 100 Milligrams of Sitagliptin and 1000 Milligrams of Metformin Hydrochloride Coated with Sustained-Release Polymer (7% w/w)

| | 100/1000 mg/ | | 50/1000 mg/ | |
|---|---|---|---|---|
| Ingredient | tablet | 100/1000% w/w | tablet | 50/1000% w/w |
| 1. Tablet Core | | | | |
| Metformin HCl | 1000 | 76.725 | 1000 | 76.725 |
| PVP K29/32 | 75.27 | 5.775 | 75.27 | 5.775 |
| Avicel PH 102 ™ | 195.50 | 15 | 195.50 | 15 |
| Silicon Dioxide | 6.517 | 0.5 | 6.517 | 0.5 |
| Sodium stearyl fumarate | 26.067 | 2.0 | 26.067 | 2.0 |
| Total Tablet cores | 1303.36 | 100 | 1303.36 | 100 |
| 2. Cellulose Acetate (CA) | | | | |
| Polymer Coating | | | | |
| CA-398-10 | 45.62 | 3.5 | 45.62 | 3.5 |
| PEG 3350 | 22.81 | 1.75 | 22.81 | 1.75 |
| Triacetin | 22.81 | 1.75 | 22.81 | 1.75 |
| Total CA SR coat | 91.24 | 7 | 91.24 | 7 |
| SR Coated Tablets | 1394.59 | 107 | 1394.59 | 107 |
| 3. Sitagliptin Coating | | | | |
| Sitagliptin phosphate | 128.52* | 9.22 | 64.26** | 4.61 |
| monohydrate | | | | |
| Propyl gallate | 1.36 | 0.098 | 0.68 | 0.049 |
| HPMC/PEG/Kaolin/dye | 130.66 | 9.37 | 65.33 | 4.68 |
| Total Sitagliptin Coat | 260.55 | 18.68 | 130.27 | 9.34 |
| Total Coated Tablet | 1655.14 | 125.68 | 1524.88 | 116.34 |

*Equivalent to 100 mg of sitagliptin free base anhydrate.
**Equivalent to 50 mg of sitagliptin free base anhydrate.

### Steps in Preparation of Example 3:

(1) metformin hydrochloride was delumped by passing it through a suitable mill;
(2) the delumped metformin and PVP dry binder powder were transferred into a granulator bowl of a high-shear granulator and granulated with water at a level of 3 to 10% of total dry powder batch size until granules were formed;
(3) the granules were dried in an oven at 50° C. to a moisture content of less than 2%;
(4) the dried granules were sized in a suitable mill to obtain a mean granule particle size of about 500-800 microns;
(5) the dried and sized granules were blended with microcrystalline cellulose (Avicel PH 102) and pre-screened (mesh #20) silicon dioxide;
(6) the pre-screened (mesh #60) sodium stearyl fumarate and blend from step 5 were blended in a suitable blender to produce the final blend;
(7) the final blend from step 6 was compressed in a rotary tablet press to produce tablets at the target weight range and hardness;
(8) the organic polymer solution was prepared by first dissolving the cellulose acetate polymer in the acetone water mixture, and then adding the PEG 3350 and triacetin to the solution while mixing until all solids were dissolved;
(9) the compressed tablet cores from step 7 were loaded into a suitable perforated side-vented coating pan with baffles fitted with single or multiple spray guns to produce a spray fan to uniformly cover the tablet bed;
(10) the tablet bed was warmed in the rotating coating pan until an exhaust temperature of 25-35° C. was reached;
(11) the average weight of warmed uncoated tablet was determined as the initial starting weight;
(12) the cellulose acetate coating solution was sprayed onto the tablet bed at a suitable spray rate and atomization pressure;
(13) spraying of the cellulose acetate polymer coating solution was continued while monitoring the tablet weight until the required weight gain was obtained; an approximate dried polymer coat weight of 91 mg was deposited over the tablet cores.
(14) spraying was stopped, and the tablets were dried and discharged from the coating pan.

(15) the sitagliptin phosphate coating solution was prepared by mixing all the excipients (except Kaolin) and sitagliptin phosphate in the required amount of purified water using a suitable homogenizer until the solids were dissolved;

(16) the pre-screened (mesh #60) Kaolin powder was added to the sitagliptin phosphate coating solution and mixed with a suitable mixer and blade until the powder was uniformly dispersed in the coating solution;
(17) the compressed tablet cores from step 7 were loaded into a suitable perforated side-vented coating pan with baffles fitted with single or multiple spray guns to produce a spray fan to cover the entire width of the tablet bed;
(18) the tablet bed was warmed in the rotating coating pan until an exhaust temperature of 40-44° C. was reached;
(19) the average weight of warmed uncoated tablet was determined as the initial starting weight;
(20) the sitagliptin phosphate coating dispersion was sprayed onto the tablet bed at a suitable spray rate and atomization pressure;
(21) spraying with the sitagliptin phosphate coating dispersion was continued while monitoring the tablet weight until the required weight gain was obtained;
(22) an approximate dried coat weight of 130 mg equivalent to 50 mg sitagliptin (as free base) or 260 mg equivalent to 100 mg of sitagliptin (as free base) was deposited over the tablet cores; and
(23) spraying was stopped, and the tablets were dried and discharged from the coating pan.

The metformin in vitro dissolution profiles (drug release rates) for several SR polymer-coated metformin tablet compositions of the present invention were measured and are shown in FIG. 1-3. All dissolution studies were conducted in USP Apparatus II at 100 rpm in 900-mL water. The three extended-release formulations produced well-differentiated metformin drug release rates with about 80% or higher of label claim being dissolved in about 4-8 hours. The duration of drug release targeted was due to a relatively narrow absorption window for metformin from the gastrointestinal tract. There is minimal absorption of metformin in the lower part of the ileum and colon, resulting in non-absorption of drug remaining in the dosage form after about 8 hours passage through the gastrointestinal tract.

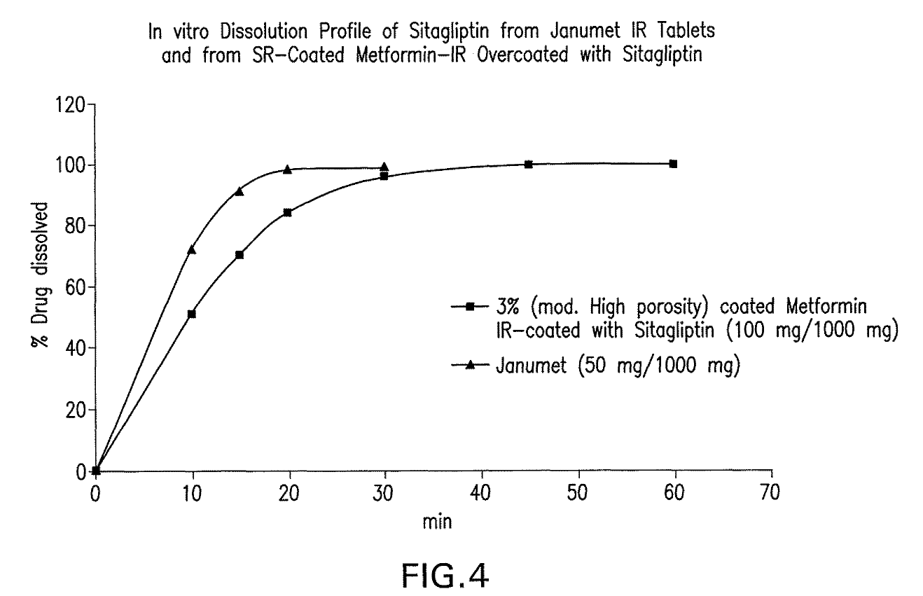

Dissolution profile of sitagliptin phosphate from the drug film layer was also measured and is shown in FIG. 4. The dissolution was found to be complete within 30 minutes and to be comparable to that of sitagliptin phosphate in JANUMET® which is a marketed fixed-dose combination of immediate-release metformin hydrochloride and immediate-release sitagliptin phosphate.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. It is intended therefore that the invention be limited only by the scope of the claims which follow and that such claims be interpreted as broadly as is reasonable.

What is claimed is:

1. A pharmaceutical composition comprising an inner core tablet composition comprising metformin hydrochloride; further comprising a coating comprising a sustained-release polymer; and further comprising a coating comprising an immediate-release composition of sitagliptin, or a pharmaceutically acceptable salt thereof, and an immediate-release polymer.

2. The pharmaceutical composition of claim 1 wherein said metformin hydrochloride is present in said inner core tablet composition in an amount of about 50 to about 80 weight percent.

3. The pharmaceutical composition of claim 1 wherein said inner core tablet composition further comprises a binding agent.

4. The pharmaceutical composition of claim 3 wherein said binding agent is polyvinylpyrrolidone.

5. The pharmaceutical composition of claim 3 additionally comprising a diluent.

6. The pharmaceutical composition of claim 5 wherein said diluent is microcrystalline cellulose.

7. The pharmaceutical composition of claim 5 additionally comprising one or two excipients selected from the group consisting of a glidant and a lubricant.

8. The pharmaceutical composition of claim 7 wherein said glidant is colloidal silicon dioxide and said lubricant is sodium stearyl fumarate.

9. The pharmaceutical composition of claim 1 wherein said sustained-release polymer is a cellulose ester selected from the group consisting of cellulose acetate, cellulose diacetate, cellulose triacetate, cellulose acetate propionate, and cellulose acetate butyrate.

10. The pharmaceutical composition of claim 9 wherein said cellulose ester is cellulose acetate.

11. The pharmaceutical composition of claim 9 additionally comprising a plasticizer.

12. The pharmaceutical composition of claim 11 wherein said plasticizer is triacetin.

13. The pharmaceutical composition of claim 11 additionally comprising a pore-forming agent.

14. The pharmaceutical composition of claim 13 wherein said pore-forming agent is polyethylene glycol 3350.

15. The pharmaceutical composition of claim 14 wherein said sustained-release polymer is cellulose acetate and said plasticizer is triacetin.

16. The pharmaceutical composition of claim 1 wherein said pharmaceutically acceptable salt of sitagliptin is the dihydrogenphosphate salt.

17. The pharmaceutical composition of claim 1 wherein said sitagliptin is present in a unit dosage strength of 50 or 100 milligrams, and said metformin hydrochloride is present in a unit dosage strength of 500, 750, 850, or 1000 milligrams.

18. The pharmaceutical composition of claim 1 wherein said immediate-release polymer is selected from the group consisting of hydroxypropylmethylcellulose, hydroxypropylcellulose, sodium carboxymethylcellulose, polyvinylpyrrolidone, and polyvinylalcohol/PEG 3350.

19. The pharmaceutical composition of claim 1 wherein said sitagliptin composition further comprises one or more excipients selected from the group consisting of a plasticizer, a dispersing agent, a colorant, and an anti-oxidant.

20. The pharmaceutical composition of claim 1 further comprising a final immediate-release film coat.

21. A method of treating Type 2 diabetes in a human in need thereof comprising the oral administration to said human a pharmaceutical composition of claim 1.
